(19) United States
(12) Patent Application Publication
(10) Pub. No.: US 2025/0032237 A1
(43) Pub. Date: Jan. 30, 2025

(54) CONTROLLED ABSORBABLE SLING SYSTEM

(71) Applicant: Azura Bio, Inc., Minneapolis, MN (US)

(72) Inventors: STASKIN David, Boston, MA (US); John NEALON, Wayzata, MN (US); Gregory KOELLER, Apple Valley, MN (US); Jan Paul ROOVERS, Soest (NL); Zeliha GÜLER, Sariyer/Istanbul (TR)

(21) Appl. No.: 18/716,871

(22) PCT Filed: Dec. 5, 2022
(86) PCT No.: PCT/US2022/051868

§ 371 Date: Jun. 5, 2024

Related U.S. Application Data
(60) Provisional application No. 63/286,057, filed on Dec. 5, 2021.

(51) Int. Cl. A61F 2/00

(52) U.S. Cl. CPC A61F 2/0045; A61F 2210/0004; A61F 2250/0023; A61F 2250/0031; A61F 2250/0051

(57) ABSTRACT

An implantable article and methods of construction and use for the treatment of soft tissue defects that impact a patient's quality of life, such as a pelvic floor injury that results in urinary incontinence. The implantable article includes an implant assembly of a support member that can have anchoring zones, accelerated ingrowth zones, and support zones. The support assembly or its zones are constructed in various ways and with various materials to selectively control the location and rate of implant absorption and tissue ingrowth. The result is the formation of a tissue bridge or hybrid tissue bridge with reinforcing members.

### PRIORITY

The present application claims the benefit of U.S. Provisional Patent Application No. 63/286,057, filed Dec. 5, 2021, which is incorporated herein in its entirety by reference.

### FIELD

The present invention relates generally to implantable devices for the treatment of incontinence and more particularly for at least partially absorbable slings for the treatment of urinary incontinence.

### BACKGROUND

Incontinence, including urinary incontinence, impacts the quality of life of people of all ages. It is common and well known that people with incontinence suffer from a loss of self-esteem, societal embarrassment, a self-imposed reduction in social and sensual activities. As a result, sufferers tend to isolate themselves, which leads to anxiety, depression, and an overall decrease in their mental health.

Incontinence is the loss of control of urine (urinary incontinence) or feces (fecal incontinence). The loss of control can be due to a number of factors, including injury, advanced age, and other illnesses impacting bone and muscle structure and integrity. While originally considered a condition associated with advanced age, the medical community now understands that individuals of all ages can suffer from incontinence. In fact, it is very common for new mothers to, at least temporarily, suffer from incontinence after the birth of their child. This is due to the stresses and strains placed on the pelvic floor structure (e.g., muscles, ligaments, and bones), the urinary system, and the gastric system while giving birth.

The urinary system consists of the kidneys, ureters, bladder, bladder outlet, and urethra. The bladder, a hollow and muscular organ, is a temporary reservoir receiving urine produced by the kidneys. The bladder temporarily stores the urine at a low pressure, which is prevented from leaving the bladder by resistance from a competent bladder outlet (i.e., the bladder neck, urethra, and muscular sphincter). During voiding, the bladder contracts sufficiently to expel the urine, while at the same time the bladder outlet relaxes and opens to prevent obstruction of the expelled urine.

The bladder and bladder outlet are supported in the pelvis by various ligaments, musculature, and fascial attachments. The urethra is connected to the bladder neck that is composed of smooth muscles forming an internal sphincter. The urethra carries the urine away from the bladder neck to the urethral opening. In the male, the urethra extends from the bladder neck to the tip of the penis—composed of the bladder neck, prostate, external sphincter, and distal urethra. In the female, the bladder neck, sphincter, and distal urethra terminate in the introitus.

While there are several causes of incontinence, mentioned above, it generally occurs due to one or more of the following conditions: 1) when the bladder is overactive and contracts without control and the sphincter muscle is unable to prevent the urine being expelled or leaked out; 2) an increase in abdominal pressure (e.g., by way of a sneeze or cough) and the sphincter is again unable to prevent the urine being expelled or leaked out; or 3) injury to the pelvic structure (e.g., a torn muscle or ligament) that results in movement of the bladder, bladder neck, or urethra from their proper anatomical location, resulting in the sphincter being unable to close properly, resulting in leaking urine. It is not uncommon for an individual suffering from incontinence to have one or more of the aforementioned conditions. This can occur alone or in combination. The etiology for an overactive bladder (urge incontinence”) may be neurological or idiopathic. The etiology of underactive outlet (“stress incontinence”) may be due to a loss of the supporting structures or intrinsic urethral dysfunction.

Incontinence is broken into five (5) types: stress incontinence, urge incontinence, mixed incontinence, overflow incontinence and functional incontinence:

Stress Urinary Incontinence (“SUI”) is an involuntary loss of urine occurring when there is a sudden increase in intra-abdominal pressure. This can occur as a result of coughing, sneezing, lifting, straining, exercise and, in severe cases, even simply changing body position.

Urge Incontinence occurs as a sudden and immediate need to urinate, resulting in a loss of bladder control prior to reaching the toilet. Urge incontinence is a result of an involuntary bladder contraction, which create the urge to urinate.

Mixed Incontinence, the most common form of urinary incontinence, is a combination of the symptoms for both stress and urge incontinence.

Overflow Incontinence consists of a constant dripping or leakage of urine out of the urethra. This occurs as a result of an overfilled bladder.

Functional Incontinence results when a person has difficulty toileting from one place to another. It is generally caused by factors outside the lower urinary tract, such as deficits in physical function and/or cognitive function.

There have been a number of treatment options available for patients suffering from urinary incontinence. The options include both external and internal treatments. External treatments consist generally of biofeedback, electrical stimulation, or Kegel exercises (repeated contraction of the pelvic floor muscles). Internal treatments consist of injectable materials (to bulk up injured or failing tissue), prosthetic vaginal-insertable devices (e.g., pessaries that hold closed or pinch the urethra), and surgery. While external treatments are generally considered a first line treatment, many patients need surgery to correct their incontinence. The most common operations for a male patient with stress incontinence are an artificial urinary sphincter or a male sling. The most prominent surgical procedure for the treatment of underactive outlet in women is a sling procedure.

Female sling procedures consist of placing a surgical device (“sling”) proximate to the urethra, bladder neck, or mid-urethra in order to stabilize or support the bladder neck, urethra, or a combination of the two. Over the years there have been several different sling procedures and slings developed. The sling procedures and slings have varied in surgical approach and variations in the design of the slings. Slings have also been made of various artificial and autologous materials and various anchoring mechanisms used to anchor the sling to the patients' abdominal muscles, ligaments, or bone.

Although serious complications associated with slings and sling surgical procedures are infrequent, they do occur. Complications have included urethral obstruction, development of a different type of incontinence, urinary retention, surgical site infection, and damage to surrounding tissue by erosion into the urinary tract or other pelvic organs. While many of these complications have been attributed to the sling procedures for the treatment of pelvic prolapse, there is still a need for a new surgical procedure to treat urinary incontinence that reduces or eliminates many of these complications.

In addition to intraoperative surgical complications during implantation, complications associated with sling procedures may be related to the anchoring points or the material utilized during the sling manufacturing process. Permanent materials have been associated with infection, pain, and difficulty with attempts to remove or modify the results.

There have been attempts at developing hybrid slings consisting of slings having one or more non-absorbable portions and one or more absorbable portions. The absorbable portion of the hybrid slings were absorbed over time. The intent of these hybrid slings was to have an absorbable portion of the sling support the urethra or bladder neck, and the non-absorbable portion of the sling act as an anchor. These early hybrid slings were an attempt to eliminate erosion beneath the urethra. Unfortunately, these slings either absorbed too quickly, causing a reoccurrence of incontinence or problems with the permanent portions of the implants.

For these reasons, there is an established need to obtain a minimally invasive yet highly effective absorbable sling and sling system that can be implanted with minimal morbidity resulting from the method of implantation or the material that is utilized.

### BRIEF DESCRIPTION OF THE DRAWINGS

FIG. 1A is a schematic view of a male urinary system.

FIG. 1B is a schematic view of a female urinary system.

FIG. 2A is top view of a sling assembly having absorbable and non-absorbable portions according to an example embodiment of the invention.

FIG. 2B is a cross section view of the sling assembly of FIG. 2A according to an example embodiment of the invention.

FIG. 2C is cross section view of another sling assembly according to an example embodiment of the invention.

FIG. 2D is a top view of a weaving configuration according to an example embodiment of the invention.

FIG. 2E is top view of a weaving configuration according to an example embodiment of the invention.

FIG. 3A is top view of sling assembly according to an example embodiment of the invention.

FIG. 3B is cross section view of the sling assembly of FIG. 2A according to an example embodiment of the invention.

FIG. 3C is cross section view of another sling assembly according to an example embodiment of the invention.

FIGS. 4A and 4B are perspective views of needles and dilators proximity to each other.

FIGS. 4B and 4C are perspective views of needles and anchored dilators proximity to each other.

FIG. 5A is a perspective view of a curved needle according to an example embodiment of the invention.

FIG. 5B is a perspective view of a generally straight needle according to an example embodiment of the invention.

FIG. 5C is a perspective view of a helical needle according to an example embodiment of the invention.

FIGS. 6A and 6B are illustrations of placement of a sling according to an example embodiment of the invention.

FIGS. 7A and 7B are illustration examples of sling configurations according to an example embodiment of the invention.

FIG. 7C is a weaving illustration according to an example embodiment of the invention.

FIG. 7D is a top view of a sling configuration according to an example embodiment of the invention.

FIGS. 7E and 7F are illustrations of placement of a sling according to an example embodiment of the invention.

FIGS. 8A and 8B are illustration examples of sling configurations according to an example embodiment of the invention.

FIG. 8C is an illustration example of sling configurations according to an example embodiment of the invention.

FIGS. 9A and 9B are illustration examples of sling configurations according to an example embodiment of the invention.

FIG. 9C is an illustration example of sling configurations according to an example embodiment of the invention.

FIGS. 10A and 10B are illustration examples of sling configurations according to an example embodiment of the invention.

FIG. 10C is an illustration example of sling configurations according to an example embodiment of the invention.

FIG. 11A is a side view of a filament showing cracks in a filament according to an example embodiment of the invention.

FIG. 11B is a cross section of a multilayered filament according to an example embodiment of the invention.

FIG. 11C is a perspective view showing a multilayered filament with molecules according to an example embodiment of the invention.

FIG. 11D is a side view of a manufacturing process of a multilayered filament according to an example embodiment of the invention.

FIG. 12A is an example of a sling configuration according to an example embodiment of the invention.

FIG. 12B is an example of a sling assembly configuration according to an example embodiment of the invention.

FIG. 12C and FIG. 12C.1 is an example of a sling assembly configuration having a biofunctional sleeve member positioned over a support member according to an example embodiment of the invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

The drawing figures are not necessarily to scale.

### DETAILED DESCRIPTION OF THE INVENTION

The following description is meant to be illustrative only and not limiting. Other embodiments of this invention will be apparent to those of ordinary skill in the art in view of this description.

The present invention is directed to various embodiments of slings suitable for and methods of implanting the slings in the treatment of female and male incontinence, including but not limited to stress urinary incontinence (“SUI”) diagnosed with urethral hypermobility or intrinsic sphincter deficiency. Although the invention, as disclosed herein generally refers to SUI, treatment of other urological disorders such as urge incontinence, mixed incontinence, overflow incontinence, functional incontinence, and other non-urological disorders, such as fecal incontinence and hernia repair, are also included within the scope of the present invention. It is contemplated that the present invention may also be utilized in conjunction with other procedures and instruments, such as, but not limited to, procedures for addressing cystocele prolapse, vaginal prolapse and anatomic hypermobility.

The various sling embodiments of the present invention may be surgically implanted by various surgical techniques and methods, including but not limited to a transvaginal approach, a transabdominal approach, and a laparoscopic approach. Additionally, the various sling embodiments may be implanted via a number of surgical approaches, including but not limited to transobturator, suprapubic, pre-pubic, or transperineal paths.

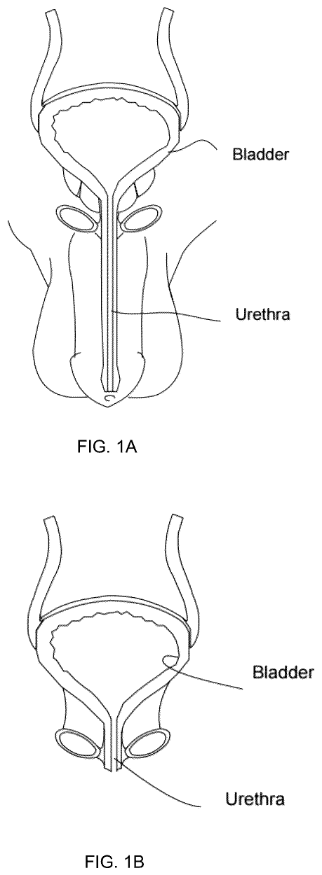

Referring to FIGS. 1A-12C.1, the present invention includes a variety of medical embodiments or assemblies 10 for treatment of various pelvic and urological disorders, as described above. The various medical assemblies 10 include a sling assembly 20 that is capable of being implanted into a patient suffering from incontinence by any of the above-described procedures. The sling assembly 20 comprises an implantable support member or portion 22 that is configured to support one or more tissues or organs to correct a pelvic condition such as urinary incontinence.

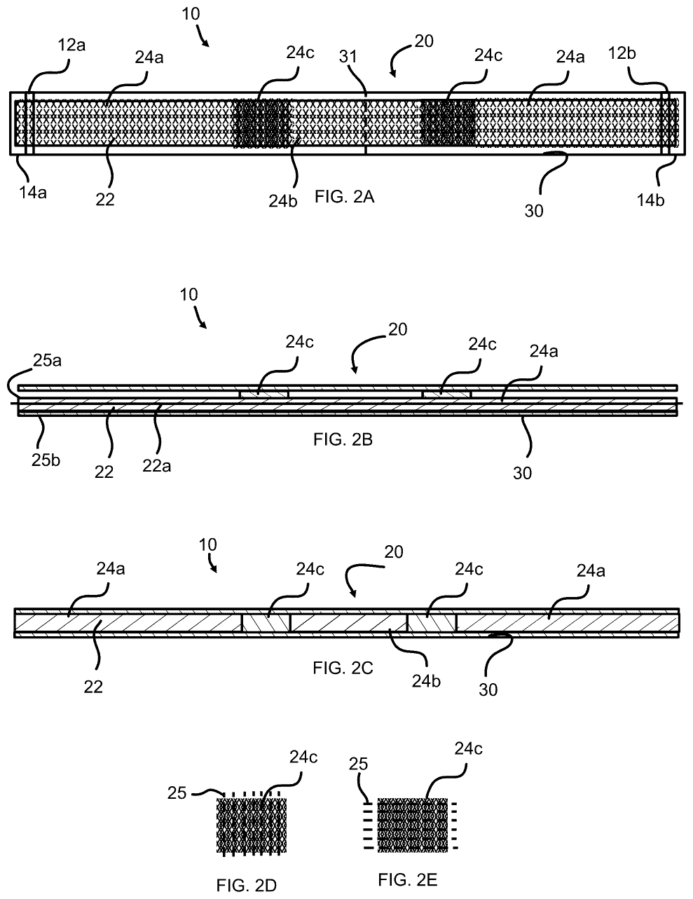

As particularly illustrated in the example embodiment of FIG. 2A, the medical assemblies 10 may also comprise a sleeve or sheath member 30 that is configured to, at least partially, enclose and protect sling assembly 20. The sleeve member 30 can protect sling assembly 20 during transportation and during implantation into the patient. The sleeve member 30 is preferably manufactured from a low friction material that enables sleeve member 30 and sling assembly 20, contained therein, to easily slide through the surgical path during implantation.

As illustrated in FIG. 2A, support member 22 and sleeve member 30 of the medical assembly 10 can be coupled or fused together at junctures 12a and 12b to aid in easing the implantation of support member or sling 22. The junctures 12a and 12b also prevent the support member or sling 22 from bunching up or sliding out of sleeve member 30. The junctures 12a and 12b can be made by heat welding, sewing, gluing, or otherwise connecting or coupling support member 22 and sleeve member 30 together. The junctures 12a and 12b are ideally located closer to opposed ends of support member 22. However, junctures 12a and 12b may be longed at any location along a length of support member 22 and sleeve member 30.

The sleeve member 30 is designed to be easily removed or separated from sling assembly 20 after implantation. The sleeve member 30 may include one or more slits, perforations, or openings 31 to permit sleeve member 30 to separate into one or more portions that can each be removed from the surgical site. The opening(s) 31 may extend perpendicular or parallel to a long axis of sleeve member 31. The junctures 12a and 12b can be cut or severed with a scalpel or scissors to aid in separating support member 22 and sleeve member 30.

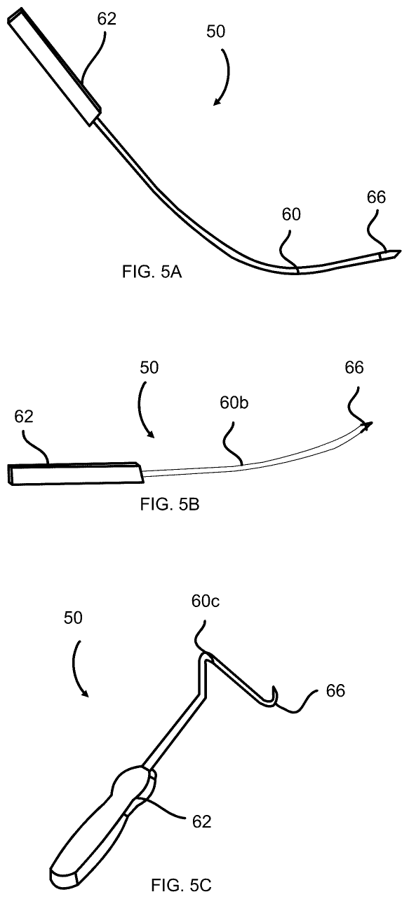

As illustrated in FIGS. 5A-5C, medical assemblies or implant 10 may further comprise one or more delivery assemblies or tools 50 that are used to during the surgical procedure to implant support assembly 20, or the combined sleeve member 30 and support assembly 20, into a therapeutic location within the patient suffering from the pelvic condition.

The composition and configuration of support member or sling 22 imparts it with the ability to strategically support desired tissues and organs, and selectively accelerating tissue ingrowth, while also possibly reducing complications. In one example embodiment, as illustrated in FIG. 2A, the configuration of support member or sling 22 of implant 10 comprises a pair of opposed anchor portions, zones, or regions 24a, a support portion, zone, or region 24b, and two or more accelerated ingrowth portions, zones, or regions 24c. Preferably, although not required, zones 24a, 24b, and 24c are also enclosed by sleeve 30.

As particularly illustrated in FIG. 2A, anchor zones 24a are proximate ends 14a and 14b of sleeve member 30. The anchor zones 24a are configured to facilitate tissue ingrowth and then be absorbed by the body. The tissue ingrowth in the anchor zones 24a form opposed ends of a tissue bridge that will ultimately extend across and support the patient's urethra or bladder neck. Anchor zones 24a may be any length, width, thickness, or configuration depending upon the particular surgical need. In one example embodiment, anchor zones 24a may have a generally greater width than other portions of support member 22 to provide a greater anchoring zone. In another example embodiment, anchor zones 24a have a width that varies along its length to accommodate particular tissue types located along the implantation pathway.

As also depicted in FIG. 2A, support zone 24b is positioned and is configured to be placed proximate to the patient's urethra or bladder neck during implantation. The support zone 24b, which may comprise a similar or different material as anchor zones 24a, is also configured to facilitate tissue ingrowth and then be absorbed by the patient's body. The support zone 24b connects the ends of the tissue bridge supporting the patient's urethra or bladder neck.

In one example embodiment of the present invention, support member or sling 22 includes one or more accelerated ingrowth zones 24c that are configured to have an increased or accelerated tissue ingrowth compared to the anchor zones 24a and the support zone 24b. The accelerated ingrowth zones 24c are generally positioned between the anchor zones 24a and support zone 24b. In this manner, accelerated ingrowth zones 24c quickly form tissue-sling connectors or connections that provide early anchoring for support zone 24b, while support member or sling 22 is forming the tissue bridge. As a result, early urinary incontinence control is achieved. Eventually, all or a portion of support member 22 is absorbed leaving the tissue bridge to support the urethra or bladder neck. Absorption of all or most of the support member or sling 22 greatly reduces previous complications.

The present invention also includes a modified implant 10 that can be used when needed, including but not limited to when there is extensive pelvic floor damage or injury. In these cases, the support member or sling 22 is configured to only be partially absorbed by the patient's body. What remains after absorption is a hybrid sling-tissue bridge comprising a tissue bridge portion that extends the full or partial length of support member or sling 22 and a reinforcing sling portion 22a. As illustrated in FIG. 2B, the reinforcing sling portion 22a may extend a full length or a portion of support member or sling 22 and is generally comprised of a non-absorbable material. The reinforcing sling portion 22a may comprise one or more fibers, filaments, or meshes.

The tissue-sling connectors or connections created by the accelerated ingrowth zones 24c can become part of the tissue bridge or the hybrid support-tissue bridge. The tissue-sling connectors or connections, comprising dense areas of tissue ingrowth that will continue to provide additional anchoring and support to the support zone 24b of the tissue bridge even after the absorption process is complete.

The support member or sling 22 of the present invention have a number of configurations. For instance, as illustrated in FIG. 2B, a single absorbable support member or mesh sling 22 may extend the length of the implant 10. One or more accelerated ingrowth zone or zones 24c can be coupled, fixed, or attached to a top surface 25a, bottom surface 25b, or both surface of the support member or sling 22. The accelerated ingrowth zone or zones 24c can be connected to the support member or sling 22 by any means, including stitching, biocompatible adhesive, seeding, or weaving. Once attached, accelerated ingrowth zones 24c define the anchoring zones 24a and support zones 24b of support member or sling 22.

In another example embodiment of the invention, as illustrated in FIG. 2C, accelerated ingrowth zones 24c are woven into or coupled to support member or sling 22. Once woven into or coupled to support member or sling 10, accelerated ingrowth zones 24c define anchor zones 24a and support zone 24b. In this example embodiment, support member or sling 22 is generally planar. The anchor zones 24a, support zone 24b, and accelerated ingrowth zones 24c can be coupled, fixed, or attached together by any means, including stitching, biocompatible adhesive, or weaving ends together.

FIGS. 2D and 2E demonstrate example weaving patterns that may be employed to create accelerated ingrowth zones 24c. In FIG. 2C, one or more tissue ingrowth acceleration fibers or filaments 25 are woven or knitted into the knit pattern of support member or sling 22. In FIG. 2C the tissue ingrowth acceleration fibers or filaments 25 are woven or knitted in a direction perpendicular to a long axis of the support member or sling 22. In FIG. 2E the tissue ingrowth acceleration fibers or filaments 25 are woven or knitted in a direction parallel to a long axis of support member or sling 22. The density and porosity of the accelerated ingrowth zones 24c can be varied by varying the knit pattern and the number of tissue ingrowth acceleration fibers or filaments 25 used. Varying the density and porosity of the accelerated ingrowth zones 24c vary the rate of tissue ingrowth and the rate of absorption.

In one embodiment, the support member or sling 22 is made of a porous material that is, as mentioned above, ideally completely or partially absorbable by a patient's body. The porous material comprises one or more woven or inter-linked filaments or fibers that form multiple fiber junctions throughout the porous material. The fiber junctions may be formed via weaving, bonding, ultrasonic welding, or other junction forming techniques, including combinations thereof. The porous material includes a plurality of pores formed during the manufacturing process that permit tissue in-growth and fixation with the porous material. The porous material may comprise a woven monofilament, knitted with a warp tricot.

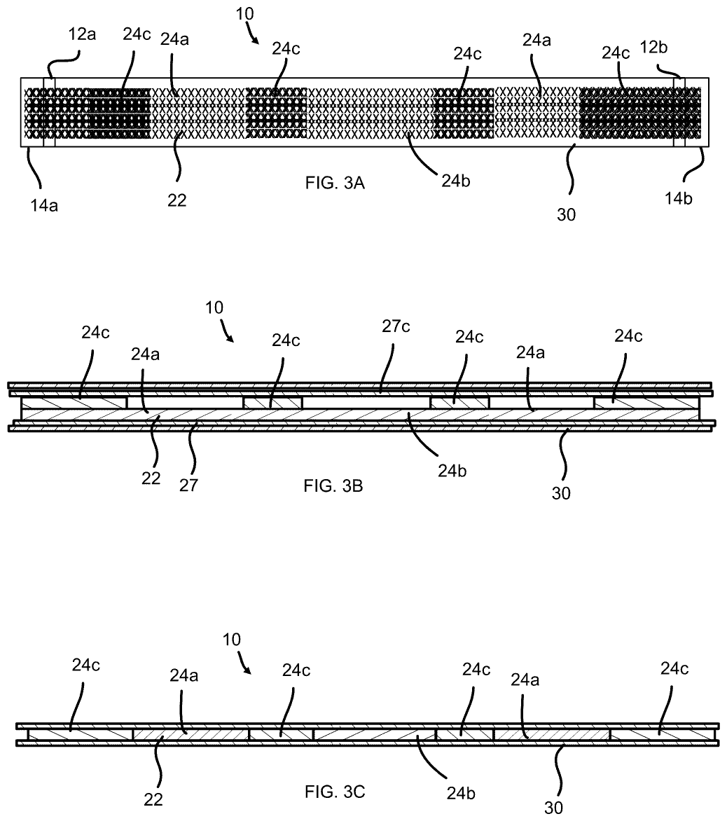

In another example embodiment of the present invention, as illustrated in FIG. 3B, the implant 10 can be made or manufactured with one or more porous materials or layers 27 positioned or connected to one or more surfaces of the support member or sling 22, anchor zones 22a, support zone 22b and/or accelerated ingrowth zones 22c. As particularly illustrated in FIG. 3B, the porous layers 27 sandwich the support member or sling 22, anchor zones 22a, support zone 22b and accelerated ingrowth zones 22c and then is enclosed by the sleeve or sheath 30. The porous layer 27 can be applied to implant 10 by an electrospinning process, including but not limited to co-axial spinning, melt spinning and/or 3D printing technique. Spun fibers or filaments can comprise of only a slow-degrading polymer, a fast-degrading polymer, or a combination of both. Additionally, the spun fibers or filaments can also comprise a non-degradable polymer.

As mentioned above, the weaving and stitch pattern/count of the various layers of the implant may be varied to provide support members or sling 22 of varying properties and characteristics. The length and thickness of the support members or sling 22 can be generally uniform or vary along the length and width of the support member or sling 22. In one example embodiment, the anchor zones 24a, support zone 24b, and accelerated ingrowth zones 24c have a height of approximately 1 cm. The length of the support member or sling 22 may be approximately 30 cm and the length of the accelerated ingrowth zones 24c may be approximately 3.0 cm. The length and height of the support member or sling 22 and its parts may vary depending upon the type of procedure being performed and the needs of the patient. For instance, the support member or sling 22 may be less than 30 cm in length for a “min-sling” procedure. Additionally, the support member or sling 22 may be slightly longer or shorter than 30 cm for a retropubic or obturator surgical approach. Further, a larger patient may need a generally larger sling 22 while a smaller patient may require a smaller sling 22.

The thickness and density of fibers or filaments forming any part of the implant 10 may be the same or vary along their length and width. For example, the anchor zones 24a and support zone 24b can consists of thicker fibers with denser patterns to allow for a slower degradation time of the implant 10 at the anchoring sites, which results in a longer mechanical support and longer time to form the tissue bridge via gradual load transfer from implant 10 to the newly formed tissue bridge. Additionally, or alternatively, fibers or filaments of accelerated ingrowth zones 24c can be thinner, and a less dense pattern to achieve faster degradation and to trigger fibroblasts and the tissue regeneration process.

In another example embodiment, the anchor zones 24a, support zone 24b, and accelerated ingrowth zones 24c comprise materials and/or combinations of materials and agents that are configured to elicit different tissue responses and then be absorbed by a patient's body. The materials or agents (and/or combinations of agents and materials) comprise pharmacologic, biologic, and cellular therapies designed to target specific steps or subsets involved in deposition of collagen and tissue in-growth. Modification of the segment to promote collagen deposition and tissue ingrowth.

The parts of the absorbable implant 10 may comprise any absorbable material. For example, naturally absorbable materials such as catgut, collagen serosa or sheep submucosa can be used. Synthetic absorbable materials such as polybutylene succinate (“PBS”), polyglycolide (“PGA”), polylactide (“PLA”), copolymers of polyglycolide and polylactide, lactomer, poly-p-dioxanone (“PDS”), PDS and Irgacare, poly-4-hydroxybutyrate (“P4HB”), and the like can also be used. The list of materials should be considered exhaustive and other materials are within the spirit and scope of the present invention.

The accelerated ingrowth zones 24c can comprise either a tissue ingrowth accelerant material or an additive tissue ingrowth accelerant. As an additive, the tissue ingrowth accelerant may be combined with the absorbable material of the accelerated ingrowth zones 24c by coating, seeding, adhering, binding, weaving, spinning, printing and the like. Any tissue ingrowth accelerating material may be used. For example, collagen material may be interwoven with the any parts of implant 10. In another example embodiment, a collagen material may be seeded in at least the accelerated ingrowth zones 24c. The collagen material may be collected from the pelvic tissue of a cadaver and combined with the accelerated ingrowth zones 24c.

Hyaluronic acid (“HA”) growth factors such as but not limited to, fibroblast growth factor (“FBG”), RGD cytokines (IL 1-beta, IL-6), tumor necrosis factor (“TNF”), platelet derived growth factor (“PDGF”), vascular endothelial growth factor (“VEGF”), and transforming growth factor (“TGF”), corticosteroids, hormones can be added to accelerated ingrowth zones 24c to facilitate fibroblasts, improve collagen formation by the fibroblasts, attenuate the inflammatory response, to avoid scar and fibrotic tissue formation.

In one example embodiment, cadaver collagen is collected, processed into a filament, and then interwoven into portions of support member or sling 22. Example weaving patterns, as discussed above, are illustrated in FIGS. 2D and 2E. However, other weaving patterns may also be used and those disclosed herein should not be considered limiting.

In another example embodiment, as mentioned above, hyaluronic acid (“HA”) can be used as an agent to accelerate the tissue ingrowth in the accelerated ingrowth zones 24c. The accelerated ingrowth zones 24c can be soaked in HA prior to implantation. Alternatively, HA can be incorporated into slow-release seeds that are then combined with the accelerated ingrowth zones 24c. The coating of the seeds is configured to release the HA over a particular period of time. While HA has been described as the tissue ingrowth accelerant agent, other materials may also be employed.

In another example embodiment, the tissue ingrowth accelerant agent is incorporated into the material of the accelerated ingrowth zones 24c during the manufacturing process. In this embodiment, as the material of the accelerated ingrowth zones 24c is absorbed it releases the tissue ingrowth accelerant agent, which in turn causes an acceleration of tissue ingrowth. This delayed release of the ingrowth accelerant agent can be accomplished by coating the sling material with a slow decaying material that upon decaying releases the ingrowth accelerant agent into the surrounding tissue, thereby increasing tissue ingrowth.

The support member or sling 22 of the present invention, can have any number of accelerated ingrowth zones 24c. As illustrated in FIGS. 3A-3C, accelerated ingrowth zones 24c can be placed at or proximate to ends of support member or sling 22. The accelerated ingrowth zones 24c proximate the ends of the support member or sling 22 provide additional accelerated tissue ingrowth and anchoring to support the support member or sling 22 while the tissue bridge is being formed.

Accelerated tissue ingrowth zones 24c can include fast degrading fibers or filaments that can be formed by changing the fiber size, weaving pattern, surface modifications via physical or chemical processes resulting surface cracks on the fibers, and/or via functionalization through coating, binding or incorporation of the molecules such as (included but not limited to) collagen, HA, dextran, cytokines (IL-6, IL1-β), RGD, growth factors (FGF, NGF, TNF, PDGF, VEGF etc.) an/or incorporation of other faster degrading material/polymer.

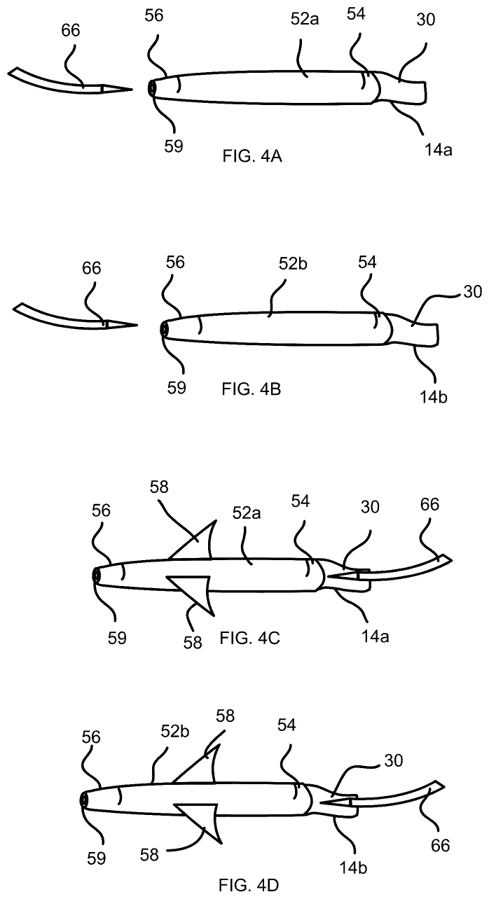

As illustrated in FIGS. 2A-2C and 3A-3C, the medical assembly or implant 10 comprises at least two opposed ends 14a and 14b, which may be part of the support member or sling 22, sleeve 30, or both. Each of the opposed ends 14a and 14b attaches to opposed dilators 52a and 52b, respectively (see FIGS. 4A and 4B). Each of the opposed dilators 52a and 52b have a first end 54 coupled to the sleeve or sheath member 30 or a combination of the support member or sling 22 and the sleeve or sheath member 30.

The dilators 52a and 52 each have a shape and configuration to dilate or open a delivery path for ease of implanting the support member or sling 22. Additionally, as illustrated in FIG. 4B, the dilators 52a and 52b may comprise one or more tissue engaging members 58 extending from them that is configured to engage tissue and to restrict or inhibit pullout of the dilators 52a and 52b after implantation. The tissue engaging members 58 may comprise barbs, tines, serrations, chevrons, ridges, spines, lips, and the like. Any configuration capable of resisting pullout may be used. The dilators 52a and 52b can be left in the patient to provide additional support or can be manufactured from an absorbable material configured to be absorbed by the patient's body after the tissue bridge is formed.

Each of the opposed dilators 52a and 52b have a second end 56 configured to mate with a distal end 66 of a delivery needle 60 (see FIGS. 5A-5C) that is used to implant the support member or sling 22. As illustrated in FIGS. 4A and 4B, second end 56 of each of dilators 52a and 52b includes an aperture 59 that is sized and shaped to quickly and securely connect to distal end 66 of delivery needle 60. This configuration may be used for instance, during a retropubic procedure where the delivery needle is initial passed through an abdominal incision to a vaginal incision where delivery needle 60 is coupled to the dilators 52a and 52b and then pulled toward or through the abdominal incision.

The delivery needle 60 can also be inserted into first end 54 of the dilators 52a and 52b to be able to drive dilators 52a and 52 into position. This configuration may be used for instance, during a transvaginal procedure where the dilators 52a and 52b are inserted via a vaginal incision.

Turning to FIGS. 5A-5C, delivery needle 60 generally has a slim and cylindrical shape to allow it to pass through the patient with ease and with minimal tissue disruption. It can be manufactured in any number of shapes depending upon the surgical technique. For instance, it can have an arc-shaped needle 60, as illustrated in FIG. 5A, a generally straight needle 60a, as illustrated in FIG. 5B, or a helical needle 60c, as illustrated in FIG. 5C. Delivery needles 60 may come in a kit as mirror pairs to allow a physician to implant each end 14a and 14b on the respective sides of the patient. Alternatively, a single delivery needle 60 may be used for the entire procedure. The single delivery needle 60 may have opposed distal ends that have different configurations for implanting the sling assembly 20 using different techniques or on different sides of the patient.

The delivery needle(s) 60 include a handle 62 that is either fixed to or detachable from the delivery needle 60. The handle 62 can be molded and may include indicia thereon that is configured to aid in identifying the particular side of implantation.

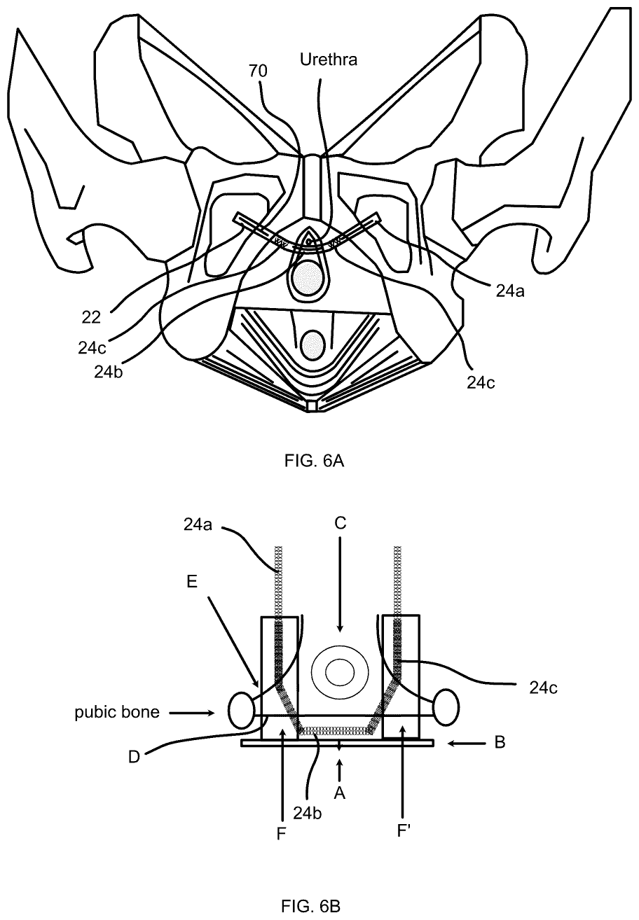

Utilizing delivery tools 60, a surgeon can surgically place implant 10 into a therapeutically effective position relative to a physiological environment intended to be supported (e.g., urethra or bladder neck). As illustrated in FIG. 6A, support zone 24b of the support member or sling 22 can be positioned below the urethra with the anchoring zones 24a positioned in an anchoring location including, but not limited to, the obturator muscles.

In one aspect of the present invention, support member or sling 22 may include a tension adjustment or control member 70 woven through it for transferring sling adjustment forces from one portion of support member or sling 22 to other portions of support member or sling 22, such as ends 14a and 14b. The tension adjustment member 70 affords effective repositioning of support member or sling 22 while avoiding undesirable permanent deformation of support member or sling 22. The tension adjustment member 70 may be manufactured from a permanent or absorbable material similar to support member or sling 22.

The tension adjustment member 70 is preferably threaded or woven along the length of support member or sling 22. The tension adjustment member 70 can be connected at one or more points along its length. For example, the tension adjustment member 70 may be affixed at junctures formed during the weaving process to distribute the anchoring points and spread the tension along its length.

Turning to the illustration of FIG. 6B, a support member or sling 22 depicts the zones 24a, 24b, and 24c of support member or sling 22 with respect to the anatomical features of the patent. Here, support member or sling 22 may have been implanted through a vaginal incision A in the patient's vaginal wall B. The anchor zones 24a, making up the arms of support member or sling 22, extend along opposed sides of the patient's urethra C. Each of the arms or anchor zones 24 is pushed or passed through the endopelvic fascia D and the pubourethral ligaments E.

As particularly illustrated in FIG. 6B, accelerated ingrowth zones 24c of implant 10 illustrated in FIG. 2A, are located in zones of increased tissue reactivity labeled as F and F′. The construction of the accelerated ingrowth zones 24c and their location with respect to the tissue or organ being supported quickly creates the formation of ingrown tissue that acts as anchor to support the tissue or organ being supported by support zone 24b of support member or sling 22. Depending up on the needs of the patient and the particular construction of implant 10, the zones of increased tissue reactivity F and F′ may extend along an entire length of support member or sling 22, in various locations along its lengths, or on particular side surfaces of support member or sling 22.

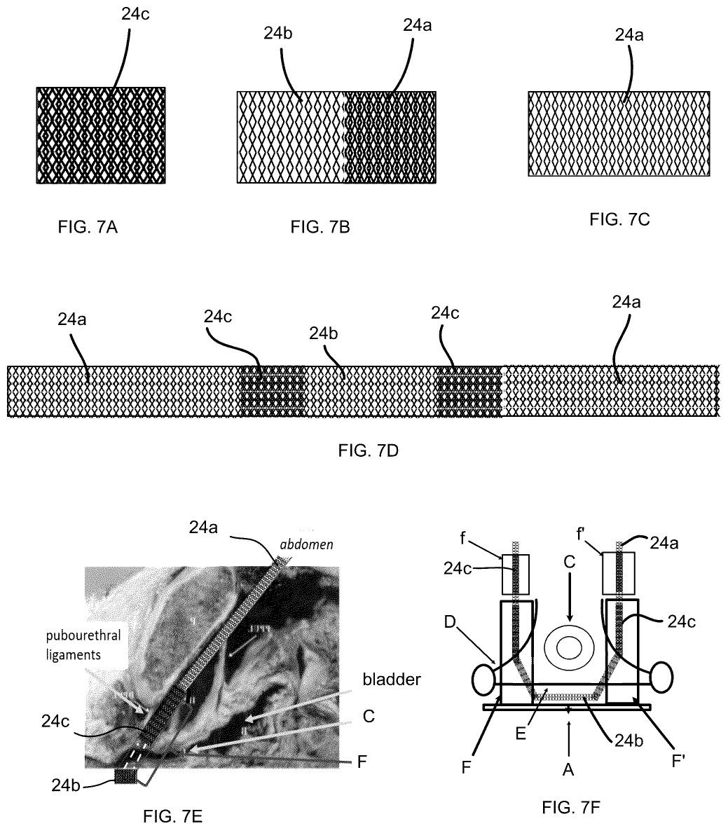

As illustrated in FIGS. 7A-7F, the zones of increased tissue reactivity F and F′ can be found in multiple locations along a length of support member or sling 22. As particularly illustrated in FIG. 7F, accelerated ingrowth zones of implant 10 in FIG. 3A are shown in their respective zones of increased tissue reactivity F, F′, f, and f′. An anatomical cross section photo is provided in FIG. 7E to aid in understanding the location of support member or sling 22 and its various zones 24a, 24b, and 24c once implanted in a patient.

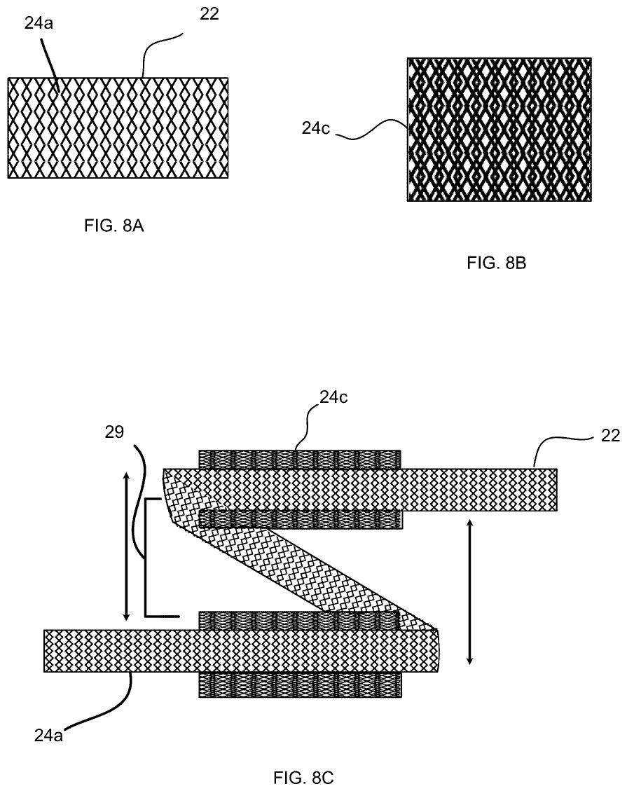

In yet another example embodiment of the present invention, as illustrated in FIGS. 8A-8C, the support member or sling 22 is manufactured with, or formed by a surgeon during the surgical procedure, a generally folded or Z-configuration or Z-zone 29. One or more accelerated ingrowth zones 24c can be coupled to, woven with, or seeded onto portions of support member or sling 22. The accelerated ingrowth zones 24c can be positioned generally with an area of the overlapping support member or sling 22 such that a larger area or an increase in accelerated ingrowth can be achieved within Z-zone 29. The location, number, and shape of folds in support member or sling 22 can vary and the description above should not be considered limiting.

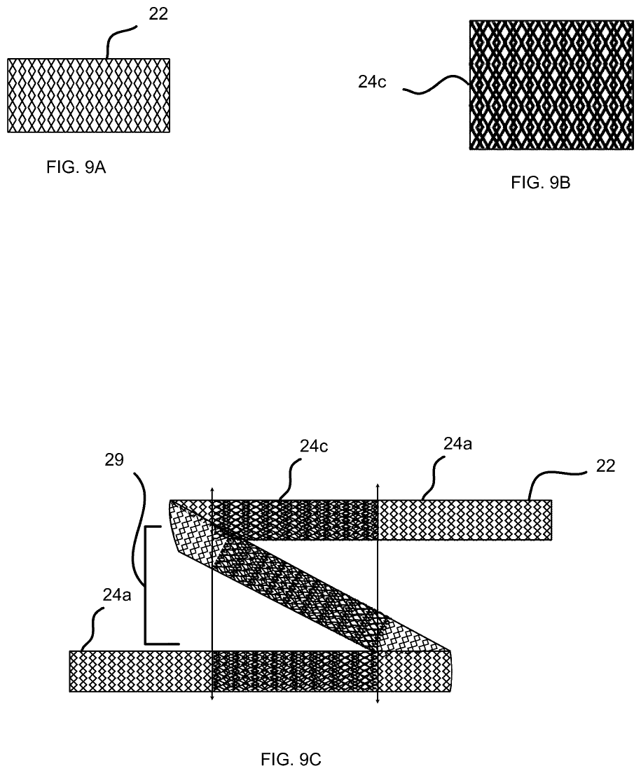

As discussed above, accelerated ingrowth zones 24c can also be formed in support member or sling 22 by simply changing one or more of the knitting parameters, densities, or the porous structure of support member or sling 22. For instance, as illustrated in FIGS. 9A-9C, accelerated ingrowth zones 24c may have the same or similar knit pattern as support member or sling 22 but have a higher overall density than the other parts of support member or sling 22. Accelerated ingrowth zones 24c can also have the same or similar knit pattern as support member or sling 22 but have a higher porosity per inch than support member or sling 22, as illustrated in FIGS. 9A and 9B.

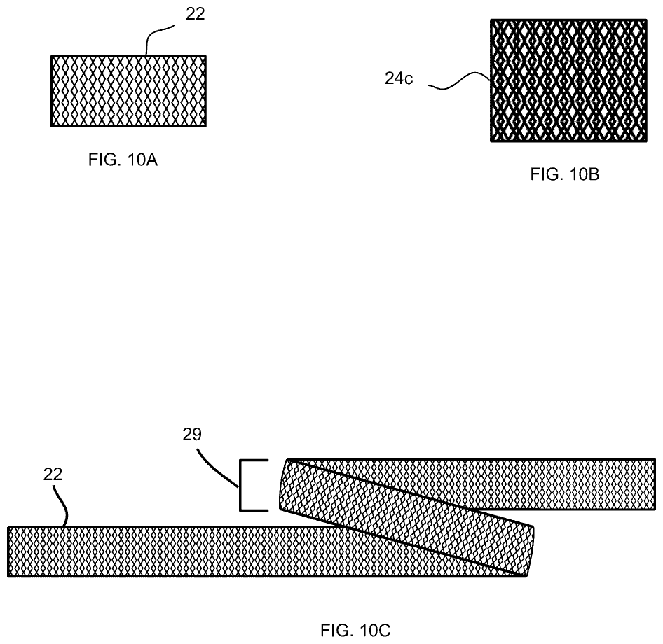

As illustrated in FIGS. 10A-10C, an entire length of support member or sling 22 can be treated, seeded, covered with, woven with fibers or filaments capable of eliciting different bioactivities, including but not limited to an accelerated ingrowth of tissue. In this embodiment, support member or sling 22 can accelerate tissue ingrowth while maintaining the same density or porosity of an untreated support member or sling 22. Like the above configurations, this embodiment includes a Z-configuration and a Z-zone 29. Again, having additional tissue ingrowth acerating materials in a localized area aids in increasing tissue ingrowth and increases the area of the accelerated tissue ingrowth.

The support member 22 can be constructed by combining electrospun and non-spun fabrics, fibers, or filaments. The number of layers can vary and any number is within the spirit and scope of the invention, but surgeons may find a two or three layered support member 22 sufficient. In another example embodiment, support member 22 can be made from one or more fibers having a co-polymer spun in or on the fibers. As with other embodiments described herein, support member can be made or constructed of fibers having different resorbable degradation profiles knitted together, different surface modifications, or different coatings. While the configuration of implants 10 have been described as having different fibers with different properties, it is also within the sprit and scope of the invention that different fibers may also contain some of the same properties.

As discussed above, the Z-configuration of support member or sling 22 allows accelerated ingrowth zones 24c to be positioned next to, proximate to, or overlap with at least a portion of an adjacent accelerated ingrowth zones 24c. In this configuration there is a localized increase in tissue ingrowth in accelerated ingrowth zones 24c. This can be particularly helpful with a patient that has thin pelvic tissue, which has been known to be difficult to treat with slings. Not only does this configuration accelerate tissue ingrowth it also can increase the size of the accelerated ingrowth zone 24c, which can aid in increasing thicker/stronger tissue to support the implant 10, and tissue or organ, during absorption and then support the tissue bridge that will then individually, or in conjunction with one or more reinforcement members, support the tissue or organ.

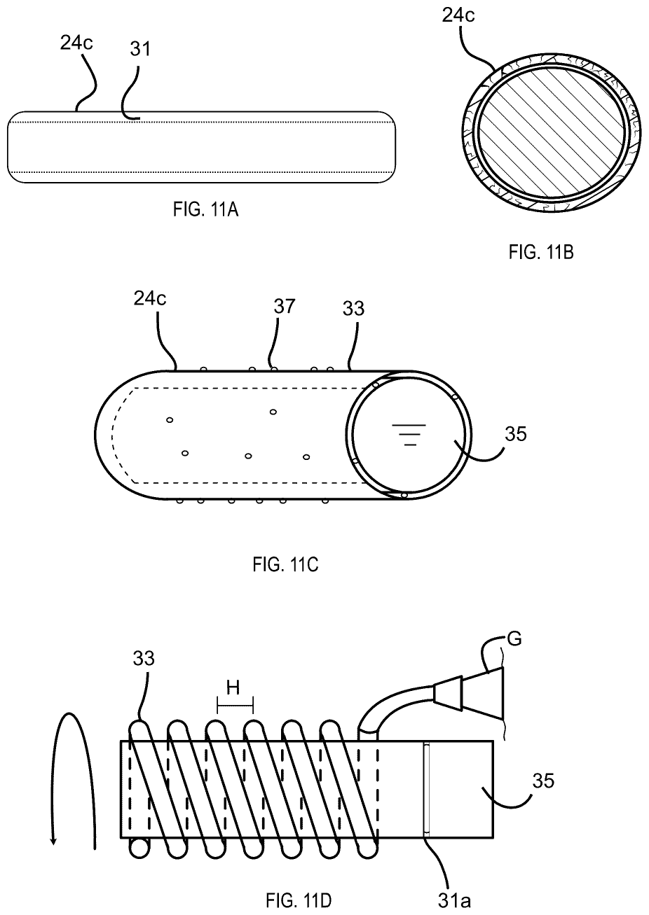

As also discussed above, accelerated ingrowth zones 24c in these configurations may have the same or different knit pattern as support member or sling 22. In another embodiment, the fibers or filaments making up the zones, 24a and 24b, and particularly 24c, may be treated with post-knit modifications to accelerate tissue ingrowth, degradation, or both. These post knit modifications can be achieved chemically, physically, or mechanically to obtain surface alterations. One such alteration is illustrated in FIGS. 11A and 11B, which show surface cracks 31 on the individual fibers or filaments of accelerated ingrowth zones 24c. The surface alteration of the individual fibers or filaments of accelerated ingrowth zones 24c results in a faster degradation to achieve accelerated tissue ingrowth and/or absorption. While the surface alterations have been shown as cracks, this should not be considered limiting but rather an example that also includes, but is not limited to, channels, grooves, and the like.

Accelerated ingrowth in accelerated ingrowth zones 24c can also be accomplished by functionalization through coating, binding or incorporation of molecules including, but not limited to, collagen, hyaluronic acid (“HA”), dextran, cytokines (IL-6, IL1-β), RGD, growth factors (e.g., FGF, NGF, TNF, PDGF, VEGF etc.). Accelerated ingrowth can also be accomplished by incorporating faster degrading material or polymers including but not limited to polylactic acid (“PLA”), polyvinyl alcohol (“PVA”), Poly lactic co glycolic acid (“PLGL”).

As illustrated in FIG. 11C, fibers and filaments of support member or sling, particularly accelerated ingrowth zones 24c, can be manufactured with a core fiber 33 covered, at least partially, by an outer cover or coating 35 made from one or more of the above materials. Additionally, or alternatively, fibers or filaments can be manufactured with molecules 37 described herein. The molecules can be coupled or bound to the covering 33, an outer surface of core fiber 33, or both. Surface alterations can also be done by using one or more chemical groups that allow biofunctionalization to be obtained (e.g., COOH groups for covalent binding of molecules via EDC-NHS chemistry) or any other surface modification resulting in more hydrophilic surfaces.

In another example embodiment, as illustrated in FIG. 11D, fibers or filaments of FIGS. 11A-11C can be manufactured by 3D printing or spun knitting, electrospinning, melt spinning, and the like. FIG. 11D illustrates a core fiber 35 having a wound or coiled cover or coating 33 applied to it by 3D printing or spun knitting G. As particularly illustrated in FIG. 11D, the 3D printing device G can be moved around core fiber 35 to apply the cover or coating 33. The spacing H between the coils can be tight, loose, or any combination thereof. A more lose coil coating 33 will increase the surface area of coating 33 thereby increasing tissue ingrowth, degradation, or both. In another example embodiment of the present invention, core fiber 35 can have one or more pores, holes, openings, or cracks 31a formed on or into it to increase tissue ingrowth, degradation, both.

Anchoring zones 24a can also comprise exclusively or partially of slow degrading fibers or filaments formed by changing the size of fiber or filaments 35, their weaving pattern and/or incorporating other slow degrading material or polymers such as but not limited to thermoplastic polyurethane (“TPU”). A combination of faster and slower degradable polymers can also be used to optimize the local host response to implant 10. A combination of different knit patterns, to influence the tissue response to implant 10.

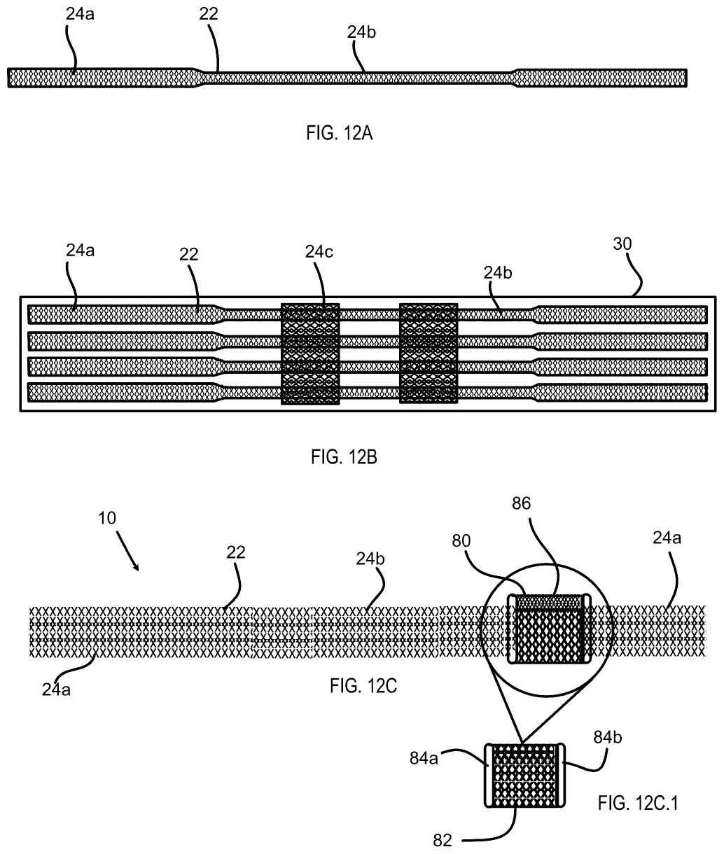

Referring to FIG. 12A, support member or sling 22 can have a varying width along its lengths. In this example embodiment, support member or sling 22 has generally wider anchor zones 24a. The support member or sling 22 then narrows or tappers toward its middle part or section of support zone 24b. Like other embodiments, support member or sling 22 can comprise of slow degrading fibers 35 formed by changing the size of fiber 35, their weaving pattern, or incorporating other slow degrading material/polymer such as TPU. Slow degrading material can be added via electrospinning (aside from the methods that are mentioned below), melt spinning, 3D printing. Fibers or filaments 35 can comprise of only slow degrading polymer and/or can be combined with any other degradable or partially degradable support member or sling 22 materials as in the form of bare fiber 35 or core-sheath fiber 33 and 35 (see FIGS. 11C and 11D).

As illustrated in FIG. 12B, a number of the support members or slings 22 can be combined with one or more accelerated tissue ingrowth materials to form accelerated ingrowth zones 24c. The construct of FIG. 12B provides a wider support across the tissue or organ being supported while also providing a wider anchoring area by way of having the widened anchoring zones 24a. Similar to other constructs of the present invention, implant 10 of FIGS. 12A and 12B, can be enclosed by sleeve or sheath 30 to aid in delivery during the surgical procedure.

Turning to FIG. 12C, the invention includes an implantable article kit that permits a surgeon to customize the implant 10 to support a tissue or organ. The implantable article kit includes the support member or sling 22 composed of one or more interconnected fibers 33/35 that can be formed in one or more mesh patterns. Similar to other embodiments, support member or sling 22 includes at least two anchor regions 24a connected to and extending away a support region 24b that is designed to support a tissue or organ.

This configuration also includes at least one biofunctional or bioactive member 80 comprising one or more interconnected filaments 33/35 that can be woven into a mesh strip or a mesh sheath 82 having opposed open ends 84a and 84b. The biofunctional bioactive member 80 or sheath 82 is positionable or connected to at least a portion of support member 22 and when combined define at least one repositionable bioactive zone 86 on support member 22.

The biofunctional or bioactive sheath 82 is configured to pass support member 22 through its open ends 84a and 84b. In this manner, a surgeon is able to move or slide biofunctional or bioactive sheath 82 along a length of support member 22. Additionally, a surgeon is able to cut or divide biofunctional or bioactive sheath 82 to create or form multiple smaller biofunctional or bioactive sheaths 82 to allow for the creation of a number of bioactive zones 86 along the length of support member 22.

There are a number of different biofunctional or bioactive member(s) 80, with each having or causing a different bioactivity within the patient or within a particular location within a patient. For instance, one biofunctional bioactive member 80 is constructed or configured to control a rate tissue ingrowth. The biofunctional or bioactive member 80 can be combined or coated with at least one tissue growth accelerant, such as hyaluronic acid, collagen, growth factors, or hormones that are disposed on at least a portion of the biofunctional or bioactive member 80. A biofunctional or bioactive member 80 can also be combined or coated with any known material capable of decreasing a rate of tissue ingrowth.

Another biofunctional or bioactive member 80 has a bioactivity configured to control an increase or a decrease in degradation of the fibers or filaments 33/35 of the biofunctional or bioactive member 80, or support member 22. By increasing or decreasing a rate of degradation of the fibers or filaments 33/35, a surgeon can control how long the implant 10, or portions thereof, is in a particular location. As mentioned above, this is important for patients with complex tissue conditions or disorders. For instance, a patient that has given birth in their lifetime, but also suffers from a disease like rheumatoid arthritis, may have thin pelvic tissue in one location and tears in another location. In this case, a surgeon can configure implant 10 to address the locations of each particular condition of the pelvic tissue.

The implantable article kit may also include one or more sleeves 30 that can at least temporarily enclose support member 22 and biofunctional or bioactive member(s) 80. Once in an operating theater, a surgeon can remove the biofunctional or bioactive member(s) 80 from sleeve 30 and prepare it for combining with support member 22. Once they are combined, a surgeon may insert the combined implant 10 back into one of the sleeves 30 for use during implantation. The sleeve 30 can then be removed once support member 22 is implanted.

For implants 10 having a Z-configuration, positioning biofunctional or bioactive member(s) 80 with the overlapping areas creates overlapping bioactive zones that are configured to increase or control a rate of a bioactivity. This configuration also permits a surgeon to have different bioactive properties within a single overlapping bioactive zone. For instance, a surgeon can construct implant 10 to have accelerated tissue ingrowth and delayed fiber or filament 33/35 degradation to permit the quickest tissue ingrowth while delaying implant 10 degradation to provide the maximum time for formation of the tissue bridge.

The biofunctional or bioactive member(s) 80 are configured to exhibit a number of bioactive conditions including, but not limited to, accelerated tissue ingrowth, accelerated fiber degradation, reduced fiber degradation, rate of an eluding material or compound, such as an antibiotic or antimicrobial. Other bioactive conditions are also within the spirit and scope of the present invention.

In another example embodiment, biofunctional or bioactive member(s) 80 can also be configured to contain or release a scar reducing agent while biofunctional or bioactive member 80, or support member 22, degrades. This property or characteristic may be particularly helpful for a patient that has had previous surgeries and has a lot of pelvic scar tissue. This implant 10 configuration allow the patient to temporarily treat their incontinence with the support member 22, while it degrades, and while the biofunctional or bioactive member 80 eludes the scar-reducing chemical or agent to reduce the amount of scar tissue. Later, a surgeon can perform another procedure with a different implant 10 configuration to strategically place a tissue bridge that will provide natural long-lasting support of the tissue or organ.

Any constructs of implant 10 discussed herein may be developed using electro spun technology having two, three, or more layers. They may also comprise one or more layers combining spun- and non-spun fiber constructs. Additionally, any of the disclosed implant 10 constructs may be spun fibers or filaments 33 and 35 combined with molecules 37 described above including, but not limited to, collagen, HA, dextran, cytokines (IL-6, IL1-β), RGD, growth factors (FGF, NGF, TNF, PDGF, VEGF etc.).

Similarly, any implant 10 constructs may have surface modification or alterations (e.g., cracks 31 or pores 31a) on a portion of or on the entire support member or sling 22. The fibers 33 and 35 of implants 10 and its support member or sling 10 may be treated with any process that alters or increases its surface to be more or less hydrophilic for fibroblasts. One example process includes plasma treatment. Any process may be employed that will affect tissue ingrowth, cell adherence and may modify immunologic response as well. These other processes include coatings of antimicrobial peptides, antibiotics, or drug eluting materials, and the like. As discussed above, any surface of constructs or implants 10 can also be altered by using one or more chemical groups that allow biofunctionalization to be obtained (e.g., COOH groups) for covalent binding of molecules via EDC-NHS chemistry or similar chemistry, or any other surface modification resulting in more hydrophilic surfaces.

The term “sling” is used generally to include a wide variety of shapes and sizes, materials, and treatments. While support member or sling 22 is preferably rectangular for treating SUI in females, other shapes are also contemplated. Depending on the treatment addressed (e.g., to provide hammock support for the bladder or bladder neck, or to address a rectocele, enterocele or prolapse), support member or sling 22 may be any of a wide variety of shapes. As an example, support member or sling 22 may be of the general shape of the slings described and shown in Moir et al., The Gauze-Hammock Operation, Journal of Obstetrics and Gynaecology of the British Commonwealth, Volume 75, No. 1, Pps. 1-9 (1968). Any shape of support member or sling 22 capable of providing a hammock support for an anatomical structure such as the urethra, bladder or the juncture between the bladder and bladder neck may be used.

The term “pore,” “crack,” or “channel” is not intended to be limiting, the pores or channels may comprise polygonal shaped holes with diagonals of 0.132 inches and 0.076 inches or variations thereof. The quantity and type of fiber junctions, fiber weave, pattern, and material type influence various support member or sling 22 properties or characteristics. In one example embodiment, a non-mesh support member or sling 22 configuration is also included within the scope of the invention. The support member or sling 22 monofilaments may be knitted with a warp tricot (as described above). The stitch count may be 27.5 courses/inch (+ or −2 courses) and 13 wales/inch (+ or −2 wales) or any variations thereof.

In another example embodiment, other substances may be included with support member or sling 22. These substances include, without limitation, drugs, hormones, antibiotics, antimicrobial substances, dyes, silicone elastomers, polyurethanes, radiopaque filaments or substances, anti-bacterial substances, chemicals, or agents, including any combinations thereof. The substances may be used to enhance treatment effects, reduce potential sling or implant 10 rejection by the body, enhance visualization, indicate proper sling 22 orientation, resist infection or other effects.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. An implantable article to support a tissue or organ, the implantable article comprising:
an elongated support member composed of one or more interconnected and at least partially absorbable fibers, the elongated support member comprising:
at least one support zone configured to support the tissue or organ;
at least two anchor zones, each of the at least two anchor zones being connected to and extending from opposed ends of the at least one support zone;
at least two accelerated ingrowth zones connected to and positioned between the at least two anchor zones and the support zones; and
wherein the at least two accelerated ingrowth zones are configured to accelerate tissue ingrowth faster than the at least two anchor zones and the support zone to provide faster anchoring of the implantable article during formation of a tissue bridge.

2. An implantable article according to claim 1, wherein the support member is at least partially enclosed in a sleeve that is removable after implantation of the implantable article.

3. An implantable article according to claim 1, further comprising a tissue growth accelerant disposed on at least a portion of the at least two accelerated ingrowth zones.

4. An implantable article according to claim 3, wherein the tissue growth accelerant comprises hyaluronic acid.

5. An implantable article according to claim 1, wherein the at least two accelerated ingrowth zones have a density greater than a density of the at least two anchor zones and the at least one support zone.

6. An implantable article according to claim 1, wherein the at least two accelerated ingrowth zones have a porosity greater than a porosity of the at least two anchor zones and the at least one support zone.

7. An implantable article according to claim 1, wherein the support member has at least one z-configuration along its length with overlapping accelerated ingrowth zones configured to increase a rate of tissue ingrowth.

8. An implantable article according to claim 1, wherein the at least two accelerated ingrowth zones comprise one or more fibers having a degradation rate faster than a degradation rate of the at least two anchor zones and the at least one support zone.

9. An implantable article according to claim 1, further comprising a retaining member connected to and extending along at least a portion of a length of the support member, wherein the retaining member is configured to be retained within and supports the tissue bridge.

10. An implantable article according to claim 2, further comprising at least one delivery tool connectable to a portion of the sleeve to deliver the support member to a therapeutic location.

11. An implantable article kit to support a tissue or organ, the implantable article kit comprising:
an elongated support member composed of one or more interconnected fibers, the elongated support member having at least two anchor regions connected to and extending away a support region;
at least one biofunctional member comprising one or more interconnected filaments being positionable or connected to at least a portion of the elongated support member and defining at least one bioactive zone on the elongated support member;
wherein the at least one biofunctional member is configured to at least increase a rate of bioactivity in the at least one bioactive zone.

12. An implantable article kit according to claim 11, wherein the biofunctional member comprises at least one accelerated ingrowth zone disposed on the support member between the at least two anchor regions and the support region, wherein the bioactivity of at least one accelerated ingrowth zone is to accelerate tissue ingrowth faster than an ingrowth at the least two anchor regions and the support region to provide faster anchoring of the implantable article during formation of a tissue bridge.

13. An implantable article kit according to claim 11, wherein the elongated support member and the at least one biofunctional member are at least partially enclosed in a sleeve that is removable after implantation of the implantable article.

14. An implantable article kit according to claim 11, further comprising a tissue growth accelerant disposed on at least a portion of the biofunctional member.

15. An implantable article kit according to claim 14, wherein the tissue growth accelerant comprises hyaluronic acid, collagen, growth factors, or hormones.

16. An implantable article kit according to claim 11, wherein the biofunctional member comprises at least one patch that can be connected to a portion of the elongated support member.

17. An implantable article kit according to claim 11, wherein the biofunctional member comprises at least one sheath slidable over a portion of the elongated support member to a desired location of increased bioactivity.

18. An implantable article kit according to claim 17, wherein the elongated support member has at least one z-configuration portion with overlapping bioactive zones configured to increase a rate of bioactivity.

19. An implantable article kit according to claim 17, wherein the elongated support member has at least one z-configuration portion with overlapping bioactive zones, with each bioactive zone having a different bioactivity, wherein different bioactive conditions are combined in a particular location.

20. A sling kit according to claim 19, wherein the bioactive zones are configured to exhibit a bioactivity selected from the group consisting essentially of accelerated tissue ingrowth, accelerated fiber degradation, reduced fiber degradation, and rate of an eluding material.
